(19) United States
(12) Patent Application Publication
(10) Pub. No.: US 2011/0229055 A1
(43) Pub. Date: Sep. 22, 2011

(54) PHANTOM FOR IMAGING APPARATUSES

(75) Inventor: Gillian Clarke, London Greater London (GB)

(73) Assignee: KING'S COLLEGE HOSPITAL NHS FOUNDATION TRUST, London Greater London, GB (GB)

(21) Appl. No.: 12/667,037

(22) PCT Filed: Jun. 23, 2008
(86) PCT No.: PCT/GB08/02148

§ 371 Date: Jul. 9, 2010

(30) Foreign Application Priority Data
Jun. 29, 2007 (GB) 0712682.4

(51) Int. Cl. G06K 9/36; A61B 6/00

(52) U.S. Cl. 382/287; 250/505.1

(57) ABSTRACT

A phantom for determining the alignment of two or more imaging apparatuses. The phantom comprises a vessel, wherein a cavity is defined by the inner surfaces of the vessel. The cavity is for holding a fluid therein. Two or more rods extend through the cavity, the rods being non-parallel with one another.

The field of this invention relates to phantoms for determining the relative alignment of imaging apparatuses. Particularly, but not necessarily exclusively, the imaging apparatuses are medical imaging apparatuses.

Medical imaging apparatuses are used to perform detailed observations of the human or animal body. Known apparatuses include, inter alia: computed tomography (CT) apparatus; single photon emission computed photography (SPECT) apparatus; positron emission tomography (PET) apparatus; and magnetic resonance imaging (MRI) apparatus. These apparatuses are used to obtain multiple images of the cross-section (transverse slices) of the body, which may be combined to build a 3D representation of the body.

Each of the imaging apparatuses has advantages and disadvantages. For example, nuclear imaging apparatuses, such as the SPECT apparatus, can be used to obtain representations of the function of the body and not just the anatomy of the body. However, SPECT apparatus generates relatively low resolution images and thus problems with localization of the elements in the images can occur; furthermore, to obtain quantitive information about a body, the images may have to be corrected for attenuation.

CT and MRI apparatuses provide representations of the anatomy of a body only. However, in contrast to the SPECT apparatus, the images are of much higher resolution, which permits a more detailed study of the anatomy of the body.

In view of the above, hybrid scanner systems have been introduced which combine (usually two) different imaging apparatuses, e.g., SPECT and CT apparatuses. By combining the imaging apparatuses, advantages of each imaging apparatus can be achieved in the single system.

One problem faced by the user of hybrid imaging systems is the accuracy of the alignment between images produced by the two apparatuses. Since, for example, one apparatus may give valuable information on localization and the other on body function, as indicated above, it is important that the images generated by the two imaging apparatuses are aligned in order that body function at a specific location can be determined accurately.

Manufacturers of hybrid imaging systems provide means to calibrate the alignment of images produced by the different imaging apparatuses. Commonly, to perform the calibration, a phantom is provided that contains radioactive source elements placed at discrete points of the phantom. Cross-sectional images of the phantom are taken by each imaging apparatus, and the positions of the cross-sectional images of the radioactive source elements in the respective images are compared with one another to check the degree of alignment between them. An example of such a phantom is disclosed in WO2005/018456. However, the performance of this phantom is limited in that it does not permit comparisons to be made of images generated across the entire phantom, since, at certain cross-sections of the phantom, no source elements are located.

Another phantom assembly is disclosed in US patent application US2005/0008126. The phantom assembly comprises, effectively, a ‘Hoffman 3-D Brain phantom’ (referred to as ‘the Hoffman phantom’ hereinafter) with a localizer surrounding the Hoffman phantom. The Hoffman phantom includes a stack of discs, each disc having a cut-out therein which, in combination with the cut-outs of the other discs, provides a water-fillable cavity that is representative in size and dimension to the human brain. The localizer comprises a sets of three indicating bars arranged in N-shapes, located around the circumference of the Hoffman phantom and between a cylindrical outer wall of the Hoffman phantom and a cylindrical outer wall of the localizer. In cross-sectional images of the phantom assembly generated by imaging apparatuses, these indicating bars show up as discrete points. Since the indicating bars are arranged in an N-shape, the distances between the points vary depending on the ‘height’, i.e. the position in the z-direction of the phantom, at which the cross-sectional images have been taken. Accordingly, cross-sectional images corresponding to the same ‘height’ position of the phantom can be determined for each imaging apparatus, and then the degree to which corresponding fixed points on these images match one another can be compared to check the alignment of the images in the cross-sectional plane of the phantom.

However, this phantom assembly is complex to manufacture and assemble in view of its high number of component parts. Furthermore, the cavity is prone to bubbles formation, when filled with fluid, due to the irregular surfaces of the cavity provided by the cut-outs in the stacked discs. The bubbles can affect the performance of the phantom and can delay the time it takes to prepare the phantom for use, if it is necessary to remove them.

Some known phantoms permit the resolution of imaging apparatuses to be determined. For example, the Jaszczak Phantom™ is provided with a plurality of rods and spheres of different sizes. By determining which sizes of rods and/or spheres can and can not be seen in cross-sectional images taken of the phantom, the resolution (or at least a range of the resolution) of the phantom can be determined. However, this phantom is complex to manufacture and assemble in view of its high number of component parts. Furthermore, it is not intended for use in determining the alignment of two or more imaging apparatuses.

### DEFINITIONS

In this application, the “z-direction of the phantom” is intended to mean the direction of a phantom along which it is intended, in normal use, that imaging systems take a succession of cross-sectional images of the phantom, and, at least in SPECT imaging, which is, or is parallel to, the rotational axis of the imaging apparatus. The cross-sectional images show 2-D representations of the phantom in the cross-sectional plane of the phantom, which can be combined to produce a 3-D representation of the phantom. The “cross-sectional plane” of the phantom is intended to mean any plane which is perpendicular to the z-direction of the phantom.

Commonly (and in some but not necessarily in all embodiments of the present invention), a phantom is cylindrical or cuboid having side walls (which are curved in the case of the cylindrical phantom) located between respective coplanar end walls. In such circumstances, the z-direction may extend between the first and second end walls, parallel to the side walls and the cross-sectional plane will extend perpendicularly to the z-direction, parallel to the end walls.

According to a first aspect of the present invention, there is provided a phantom for determining the alignment of two or more imaging apparatuses, the phantom comprising:
a vessel, wherein a cavity is defined by the inner surfaces of the vessel, the cavity being for holding a fluid therein;
two or more rods extending through the cavity, the rods being non-parallel with one another.

According to a second aspect of the present invention, there is provided a phantom for determining the alignment of two or more imaging apparatuses, the phantom comprising a housing, and two or more conduits extending through the housing, the conduits being for holding a fluid therein, the conduits being non-parallel with one another.

The rods/conduits are for determining relative alignment of cross-sectional images of the phantom, generated by each imaging system, in both the z-direction of the phantom and in the cross-sectional plane of the phantom.

The phantom of the second aspect is very similar to the phantom of the first aspect but it can be considered a ‘negative’ of the phantom of the first aspect: fluid located in the conduits may take substantially the same form as the material of the rods, and fluid located in the cavity may take substantially the same form as the material of the housing. These two aspects may provide alternative solution to problems presented by the prior art, by using generally the same inventive concepts, as is evident from the discussions below.

According to a third aspect of the present invention, there is provided a method of determining the alignment of two or more imaging apparatuses, the method comprising the steps of:
providing a phantom according to the first or second aspect of the present invention;
generating respective cross-sectional images of the phantom using each imaging apparatus, the rod or conduit cross-sections being shown in the images; and
determining, from the positioning of the rod/conduit cross-sections, the relative alignment of the images in the z-direction and cross-sectional plane of the phantom.

By having the rods or conduits non-parallel with one another, the separation of their cross-sections as shown in the images will be dependent on the positions at which the images have been generated in the z-direction of the phantom. The rods or conduits will move toward and away from each other as they extend in the z-direction of the phantom. Thus, cross-sectional images generated by two different imaging apparatuses at the same position in the z-direction of the phantom can be determined, and then these images can be compared with one another to determine whether they are aligned in the cross-sectional plane of the phantom. Specifically, the alignment of the images in the cross-sectional plane of the phantom can be determined by checking the degree to which the positions of corresponding rod or conduit cross-sections in the images match. This may be a visual check, or the positions of the cross-sections may be analysed and compared automatically, e.g. using scanning and data processing apparatus.

A visual check is advantageous, as it may allow the end-user of the system (e.g. a hospital technician) to determine quickly whether there is an alignment problem, and advise e.g. the manufacturer of the system of the problem without delay and without the need for other, potentially expensive, equipment. Nevertheless, an automatic scanning and data processing apparatus may be particularly advantageous in order to quantify misalignment of the imaging apparatuses and/or, if it is integrated with automatic adjustment apparatus, to adjust the positioning and/or set-up of the imaging apparatuses automatically in order to correct for misalignment.

As can be seen, the same rods or conduits can be used to determine the alignment of images produced by the imaging apparatuses in both the z-direction and in the cross-sectional plane of the phantom. Furthermore, by extending from one end of the cavity or housing to the other, the alignment of images generated across substantially the entire phantom in the z-direction may be considered.

By positioning the rods in the cavity, or positioning the conduits in the housing, construction of the phantom may be much easier than if, for example, the rods or conduits were located in a separate compartment external to the cavity or housing. Furthermore, by placing the rods in the cavity, or conduits in the housing, the alignment of the images may be determined across substantially the whole cross-section of the cavity or housing, which may have a shape that corresponds more closely to a region of interest of a body to be imaged by the system.

Preferably, the vessel comprises sidewalls located between first and second end walls, wherein the cavity is defined by the inner surfaces of the sidewalls and end walls, and the rods extend from the first end wall to the second end wall through the cavity. The rods may each comprise a bore extending along the elongation direction of the rod. Preferably, the bores have a diameter of less than 2 mm, preferably between 1 mm and 2 mm. The bores may show up as small dots in the cross-sectional images of the rods, which may allow for more precise determination of the position of the rods. The bores may comprise fluid therein. The fluid may be radioactive, and sealed within the bore. Instead of radioactive fluid, other types of radioactive sources may be sealed in the bores.

Preferably, the housing comprises a solid block of material. The block of material may have first and second end faces and side faces extending therebetween. The conduits may be provided by bores in the block of material. The conduits are preferably straight, and preferably they each define an elongated, cylindrical cavity. Construction of the phantom of the second aspect may be relatively straightforward. Preferably, the material is light material, e.g. polystyrene, to reduce the overall weight of the phantom, enabling easier handling. By filling only the conduits with fluid, rather than filling a cavity with fluid (bar the rods extending through the cavity), the phantom of the second aspect of the present invention may be lighter than the phantom of the first aspect.

Preferably, the vessel or housing has a circular cross-section (i.e. is cylindrical) or has an elliptical cross-section. An elliptical cross-section is more preferable when mimicking of the cross-section of a human body is desired. Preferably, one of the end walls or faces provides a means for placing fluid into the vessel or conduits. For example, an end wall may be removable and resealable with the vessel, or an end wall or end face may contain one or more sealable aperture etc., to provide fluid access.

Preferably, more than two, more than three, more than four or more than five rods or conduits are provided. Preferably, all the rods or conduits are non-parallel with one another and preferably all the rods or conduits extend through the cavity or housing. The more rods or conduits that are provided, the more accurate the determination of misalignment between imaging apparatuses can be, as an average degree of misalignment can be obtained using a greater number of rods or conduits. Furthermore, any distortion in the image can be easier to observe and assess if there are more rods or conduits, since there can be greater coverage of the field of view of the imaging apparatuses. Still furthermore, a plurality of rods or conduits may be used to determine more accurately the spatial resolution of the imaging apparatuses, as discussed further below.

Preferably, one or more of the rods or conduits extends partially or entirely through a core region of the cavity or housing. The core region may extend across the full extent of the cavity or housing in the z-direction of the phantom, but may extend only partway across the cavity or housing in the cross-sectional plane of the phantom. Preferably, if the cavity or housing is cylindrical, the core region is concentric with the cylindrical cavity or housing and extends in the cross-sectional plane of the cavity or housing to no more than 50 or 60% of the diameter of the cavity or housing. By extending through the core region of the cavity or housing, the alignment of the images may be determined at a region of the phantom which corresponds more closely to a region of interest of a body to be imaged by the apparatus.

Preferably, the inner surfaces of the vessel (i.e. the walls of the cavity), or the inner surfaces of the conduits, are smooth. For example, the cavity may be cylindrical, so that it has smoothly curving side walls. Accordingly, the cavity is less likely to produce bubbles when it is filled with fluid and therefore the phantom may be prepared for use more quickly.

Preferably, the fluid placed in the cavity or conduits is a radioactive fluid, particularly for use with SPECT or PET imaging apparatuses.

Preferably the imaging apparatuses are SPECT and CT imaging apparatuses, although it is conceived that the phantom could be used also with at least PET and CT imaging apparatuses and PET and MRI imaging apparatuses.

Preferably, the rods or conduits are distinguishable from one another in the images taken by the imaging apparatuses. For example, the rods or conduits may have different cross-sectional shapes and/or sizes, so that their cross-sections as shown in the images look different from one another. This may be advantageous particularly if there would otherwise be one or more lines of symmetry in the images. Having distinguishable rods or conduits in such circumstances will ensure that the images are not unknowingly ‘out of phase’, e.g. by 180 degrees.

Furthermore, if a plurality of rods or conduits are provided with different cross-sectional sizes, they may be used to give an indication of the spatial resolution of the imaging systems, since rods or conduits with cross-sectional sizes below the spatial resolution of the imaging systems will not be shown in the images. Preferably, a rod or conduit is provided with a cross-sectional size at the expected spatial resolution limit of one or both of the imaging apparatuses, to determine whether the imaging apparatuses achieve this spatial resolution. If a number of rods or conduits are provided with different cross-sectional sizes around the expected spatial resolution limit of the imaging apparatus, a fairly precise range for the spatial resolution of the imaging apparatus may be determined, wherein the limits of the range correspond to the cross-sectional sizes of the largest rod or conduit that cannot be seen and the smallest rod or conduit that can be seen.

### BRIEF DESCRIPTION OF THE DRAWINGS

Embodiments of the present invention will now be described by way of example only, with reference to the accompanying drawings, in which:

FIG. 1 shows an isometric view of a phantom according to a first embodiment of the present invention;

FIG. 2 shows a side view of the phantom of FIG. 1;

FIG. 3 shows a plan view of the phantom of FIG. 1;

FIG. 4 shows an end view of the phantom of FIG. 1;

FIG. 5 shows another oblique view of the phantom of FIG. 1;

FIG. 6 shows representative images generated by an imaging apparatus along the z-direction of the phantom;

FIG. 7 shows fused transaxial images of the phantom of FIG. 1 taken by CT and SPECT imaging apparatuses;

FIGS. 8a, 8b show representative images of the phantom generated by CT and SPECT imaging apparatuses respectively and FIG. 8c shows an amalgamation of the images of FIGS. 8a and 8b;

FIG. 9 shows a representative image of another amalgamation of two images generated by CT and SPECT imaging apparatuses;

FIG. 10 shows a side view of a phantom according to a second embodiment of the present invention;

FIG. 11 shows a representative image of the phantom of FIG. 10 generated by SPECT imaging apparatus; and

FIG. 12 shows a side view of a phantom according to a third embodiment of the present invention.

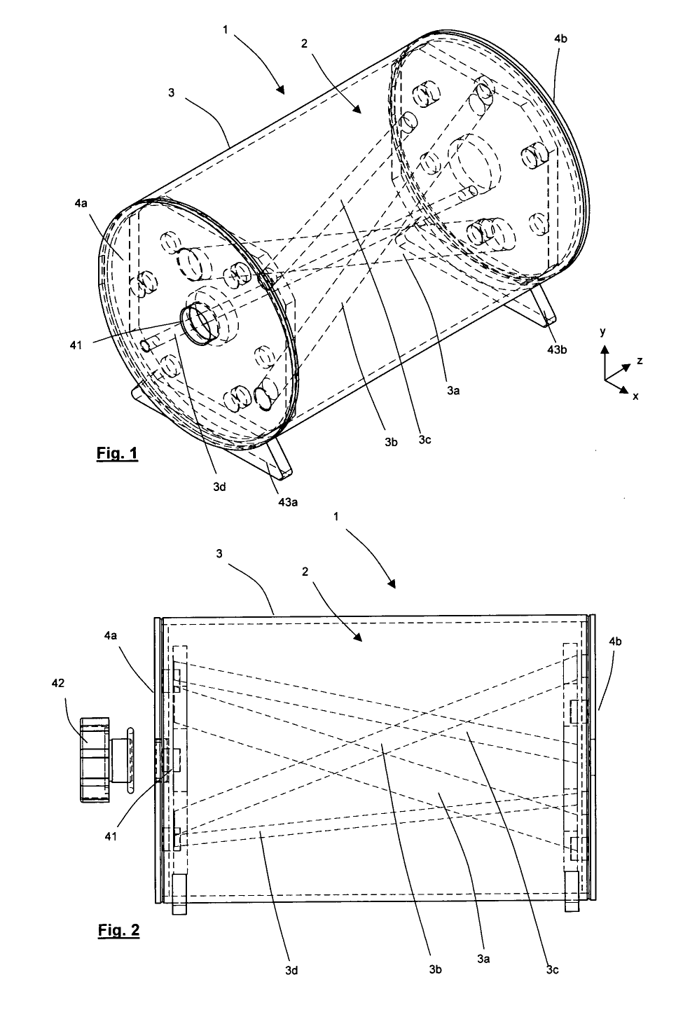

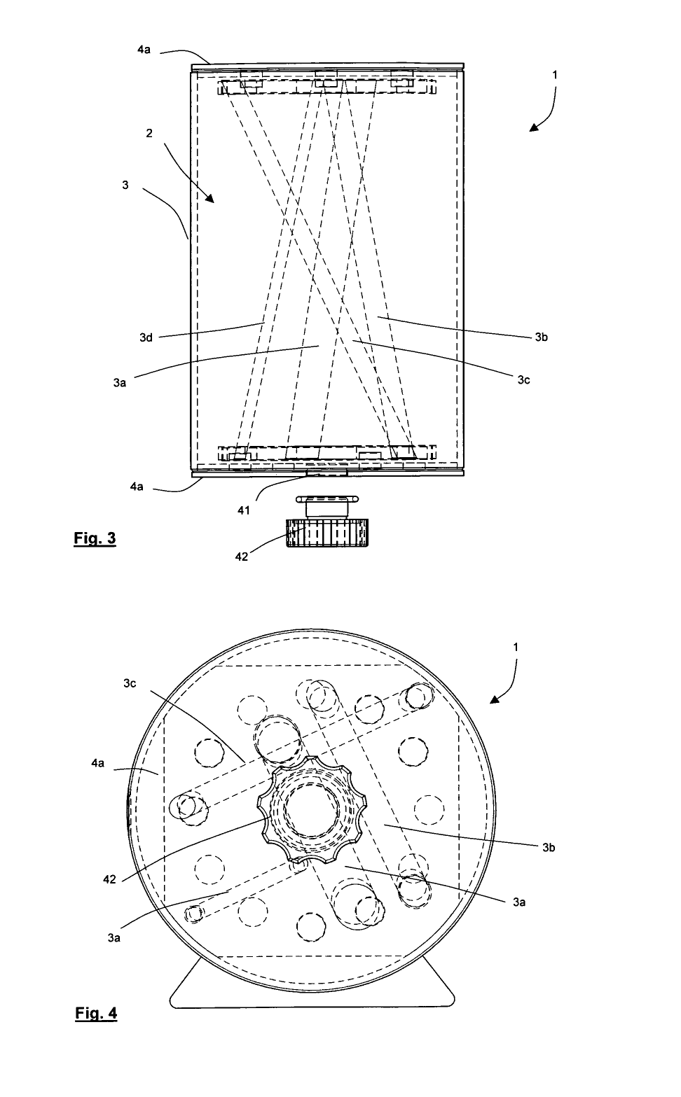

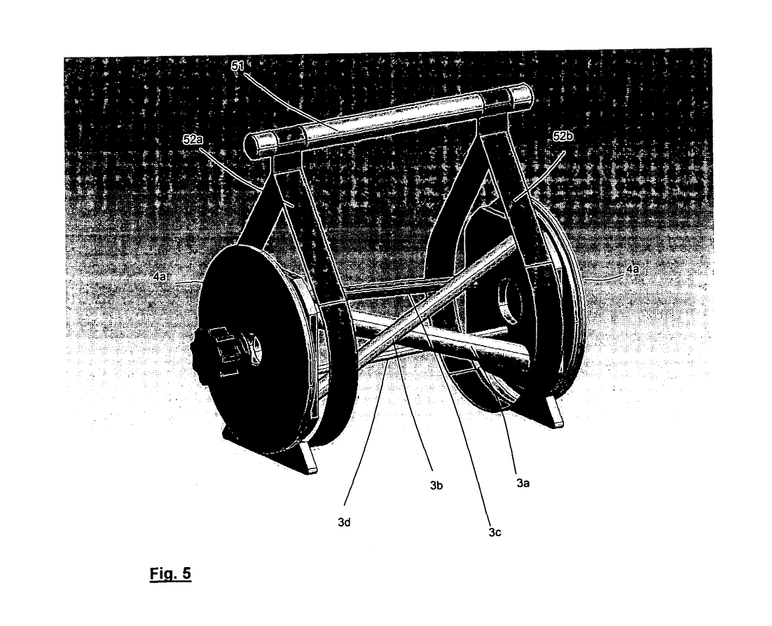

A phantom 1 according to a first embodiment of the present invention is shown in FIGS. 1 to 5. The phantom 1 has a cylindrical outer casing 2 comprising curved sidewalls 3 extending, in the z-direction of the phantom 1, between first and second end walls 4a, 4b. FIGS. 1 to 4 are line drawings and FIG. 5 is a CAD (computer-aided design) drawing. In FIGS. 1 to 4, features of the phantom 1 which may be enclosed by the outer casing 2 in normal use are represented using dotted lines.

The walls 3, 4a, 4b of the outer casing 2 define an internal cavity for holding radioactive fluid. Fluid can be dispensed into the cavity via an aperture 41 provided through the first end wall 4a. A cap 42 is provided to releasably close the aperture 41 in order to seal the fluid within the cavity.

The phantom 1 is supported by two support elements 43a, 43b located proximate the first and second end walls 4a, 4b respectively, and underneath the phantom 1.

In this embodiment, the phantom 1 comprises four rods 3a-3d which extend through the cavity between inner surfaces of the first and second end walls 4a, 4b. The rods 3a-3d are cylindrical. The rods 3a-3d are non-parallel with one another and have different cross sectional areas in the cross-sectional plane of the phantom (i.e. a direction parallel to the x-, y-plane of the phantom as shown in FIG. 1). In the Figures, the rod marked “3a” has the largest cross-sectional area and the rod marked “3d” has the smallest cross-sectional area. None of the rods are parallel with the z-direction of the phantom 1 in this embodiment.

With reference to FIG. 5, a handle may be provided for lifting and positioning the phantom 1. The handle may comprise a bar 51 connected to the phantom via two slings 52a, 52b proximate either end of the bar 51, the slings 52a, 52b extending around the casing 2 (not shown in FIG. 5) of the phantom 1.

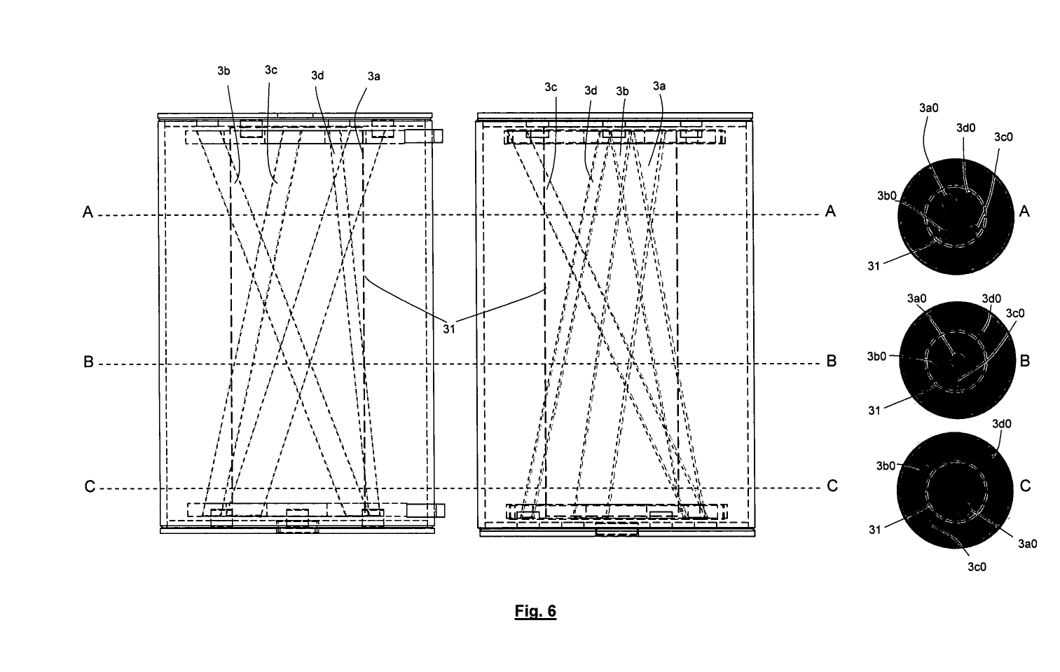

FIG. 6 shows side and plan views of the phantom 1 side by side, along with representative cross-sectional images A, B and C of the phantom 1, generated by an imaging apparatus at different positions along the z-direction of the phantom. The different cross-sectional planes along the z-direction of the phantom 1 at which images A, B and C have been generated are indicated by dotted lines A-A, B-B and C-C respectively.

As can be seen in FIG. 6, the positions of the cross-sections 3a0-3d0 of the rods 3a-3d that are shown in each image A, B and C are different, both relative to each other and relative to the peripheral edge of the image. This is as a result of the rods being non-parallel with each other and non-parallel with the z-direction of the phantom 1. With this arrangement, the positions of the rod cross-sections 3a-d would be different for any images generated at different positions along the z-direction of the phantom 1. As can be seen, all of the rods extend at least partially through a core region of the cavity which is indicated by dotted lines 31, the core region 31 is shown to extend in this embodiment between the two side walls 4a, 4b and across 50% of the cross-sectional diameter of the cavity.

Since the rod cross-sections 3a0-3d0 are positioned differently in each image generated at different positions along the z-direction of the phantom 1, the position along the z-direction of the phantom at which images have been generated can be determined. Precise positions along the z-direction can be determined for example by calculating the coordinates of the rod cross-sections 3a0-3d0 in each image. The coordinates can be entered into processing apparatus (not shown) comprising information about the correlation between the coordinates of the rod cross-sections and positions along the z-axis of the phantom 1. The processing apparatus may be automated and may comprise e.g. a computer and associated software.

Since the position along the z-direction of the phantom 1 at which images have been generated can be determined, images taken at the same position along the z-direction of the phantom can be determined for two different imaging apparatuses, e.g. SPECT/CT, and the alignment of the images, and thus the imaging apparatuses, in the cross-sectional plane of the phantom 1, can be compared.

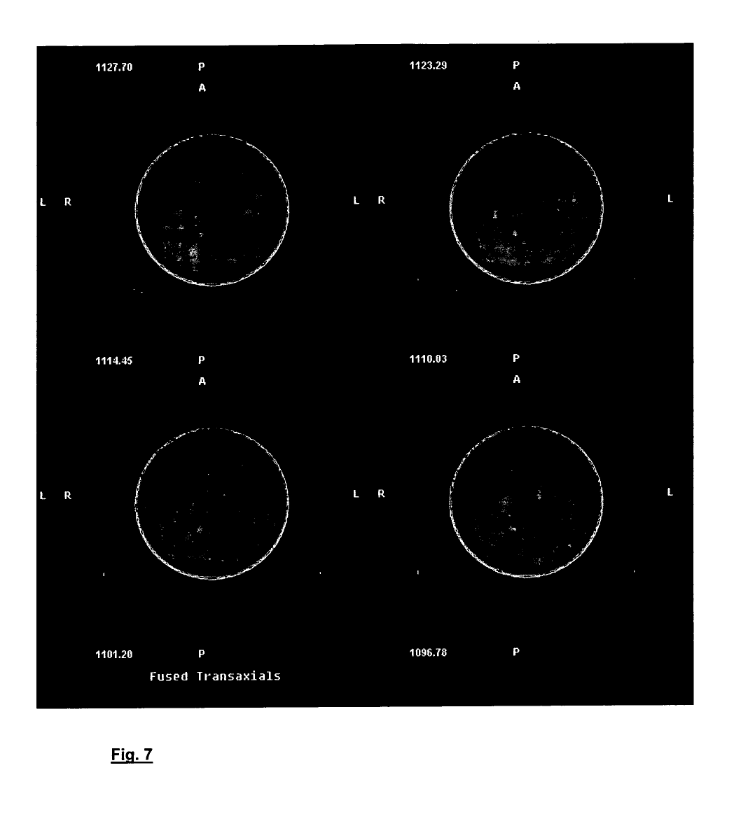

Example ‘fused transaxial’ images (two amalgamated images taken by SPECT and CT imaging apparatuses respectively) generated for the phantom 1 are shown in FIG. 7. The rod cross-sections as produced by each imaging apparatus can be seen as circular disks in each image. The light coloured disks are generated by the CT imaging apparatus and the darker coloured disks (appearing almost as shadows) are generated by the SPECT imaging apparatus (The SPECT imaging apparatus produces dark colours corresponding to regions of the phantom which are not radioactive).

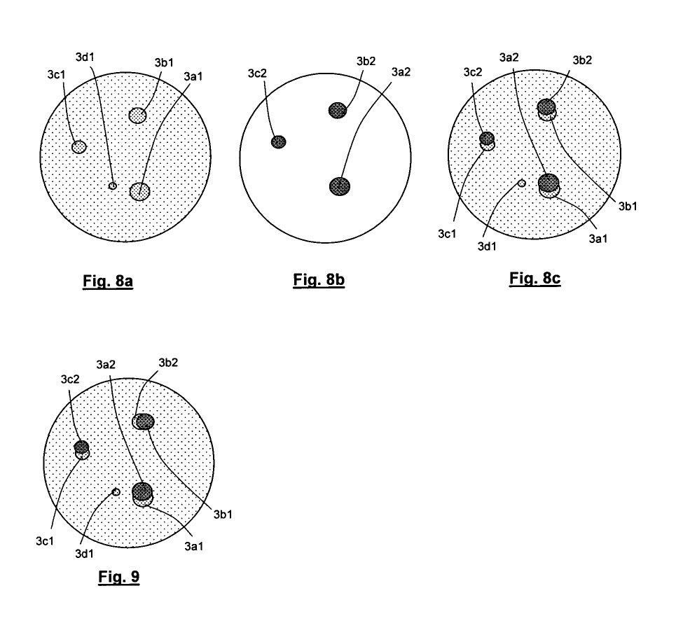

To aid understanding of FIG. 7, FIGS. 8a to 8c are provided, which are simple representations of the images produced by the CT imaging apparatus (FIG. 8a), the SPECT imaging apparatus (FIG. 8b) and of these two images amalgamated (FIG. 8c). In FIG. 8a, the rod cross-sections 3a1-3d1 for all four rods 3a-3d can be seen. However, in FIG. 7b, the rod cross-sections 3a2-3c2 for only three rods 3a-3c can be seen. This is due to the resolution of the SPECT imaging apparatus being lower than the resolution of the CT imaging apparatus and being too low to distinguish the small cross-sectional area of the smallest rod 3d. By knowing the cross-sectional areas of the rods 3a-3d, and by determining which rod cross-sections can and cannot be seen in an image produced by one or both of the imaging apparatuses, a range for the resolution of that imaging apparatus can be determined.

Referring to FIG. 8c, it can be seen that the rod cross-sections 3a2-3c2 generated by the SPECT imaging apparatus are offset from the rod cross-sections 3a1-3c1 generated by the CT imaging apparatus. A simple glance at this amalgamated image allows a person to see immediately that the imaging apparatuses are misaligned. Furthermore, by determining the distances and directions to which the cross-sections 3a1-3c1, 3a2-3c2 are offset, the degree and direction to which the imaging apparatuses are misaligned can be calculated, and appropriate action to align the imaging apparatuses can be taken.

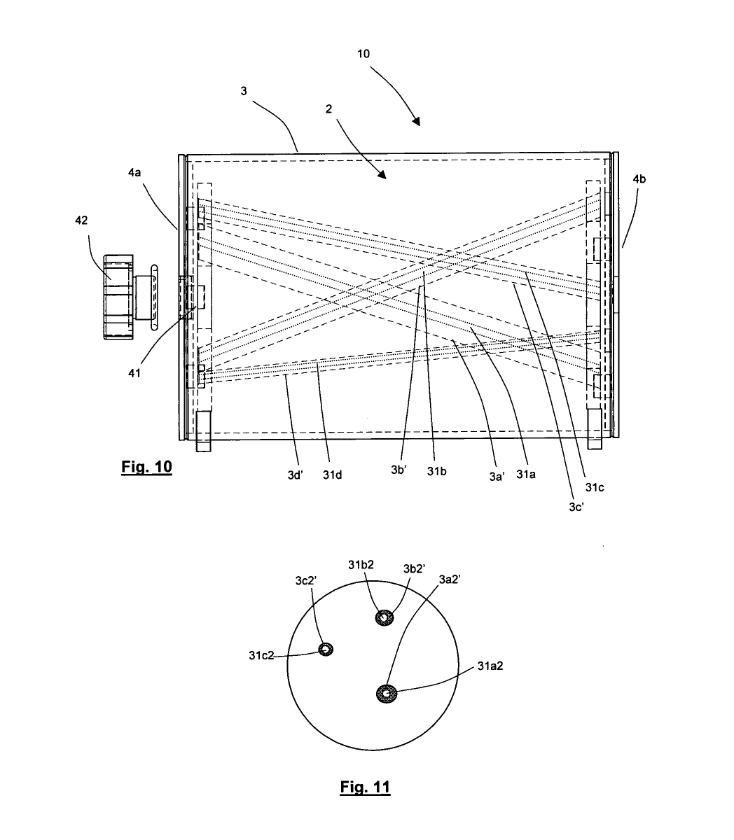

FIG. 9 shows another representation of an amalgamated image produced by CT and SPECT imaging apparatuses, similar to FIG. 8c. As can be seen, the rod cross-sections 3a2-3c2 generated by the SPECT imaging apparatus are offset from the rod cross-sections 3a1-3c1 generated by the CT imaging apparatus. However, the directions in which the rod cross-sections 3a1-3c1, 3a2-3c2 are offset is not consistent. Rod cross-sections 3b1 and 3b2 are offset in a generally left-right direction as seen in FIG. 9, whereas rod cross-sections 3a1 and 3a2, and 3c1 and 3c2, are offset in a generally up-down direction as seen in FIG. 9. This inconsistency is indicative of image distortion. A simple glance of this amalgamated image would allow a person to determine that image distortion exists, in addition to image misalignment. Furthermore, by determining the distances and directions to which the cross-sections 3a1-3c1, 3a2-3c2 are offset, the degree and direction to which the imaging apparatuses are misaligned and produce distorted images can be calculated, and appropriate corrective action to align the imaging apparatuses and correct for distortion can be taken. To allow a more detailed analysis of distortion, the phantom 1 may be provided with more rods.

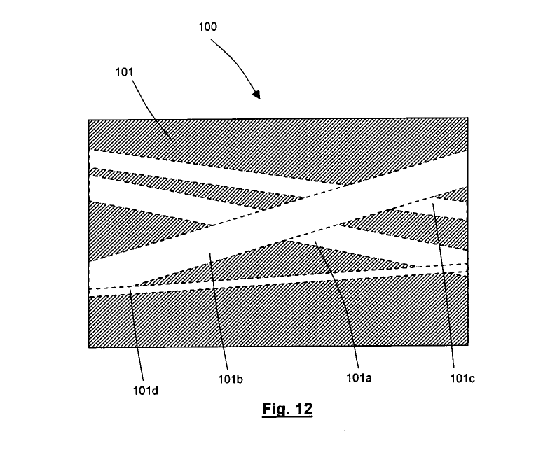

FIG. 10 shows a phantom 10 according to a second embodiment of the present invention. The phantom 10 is identical to the phantom 1 of the first embodiment except that each of the rods 3a′-3d′ has a respective bore 31a-31d extending through it, in its elongation direction. Each bore 31a-31d is for accommodating a fluid, e.g. a radioactive fluid. The bores extend centrally along the entire length of the rods 3a′-3d′, making the rods 3a′-3d′ tubular in nature. With reference to FIG. 11, which shows a SPECT cross-sectional image of the phantom 10 similar to the image shown in FIG. 8b, by having the bores 31a-31d in the rods 3a′-3d′, with radioactive fluid therein, dots 31a2-31c2 show up in the middle of the rod cross-sections 3a2′-3c2′ in the image. For PET imaging, the fluid preferably has f-18 radioactivity. The dots, which are of course smaller than the entire cross-sections of rods, permit more precise positional information about the rods to be determined from the images.

FIG. 12 shows a phantom 100 according to a third embodiment of the present invention. The phantom 100 is essentially a ‘negative’ of the phantom 1 of the first embodiment, in that, instead of a cavity, a housing 101 consisting of a solid block of material (e.g. polystyrene with a waterproof coating) is provided, and instead of rods, cylindrical bores 101a-101d are provided in the solid material, for holding radioactive fluid therein. FIG. 12 is a cross-sectional image of the phantom and, although the entire lengths of all of the bores 101a-101d would not normally be seen in such a cross-sectional image, they have been included in FIG. 12 to aid understanding of this embodiment. The phantom 100 provides a means for determining the alignment of two or more imaging apparatuses, and the resolution of the imaging apparatuses, generally as described above with respect to the first embodiment. By having fluid in the bores 101a-101d only, however, the phantom 100 of the third embodiment may be considerably lighter than the phantom 1 of the first embodiment.

1. A phantom for determining the alignment of two or more imaging apparatuses, the phantom comprising:
a vessel, wherein a cavity is defined by the inner surfaces of the vessel, the cavity being for holding a fluid therein;
two or more rods extending through the cavity, the rods being non-parallel with one another.

2. The phantom of claim 1, wherein more than two rods, more than three rods, more than four rods or more than five rods are provided that extend through the cavity.

3. The phantom of claim 2, wherein all the rods are non-parallel with one another.

4. The phantom of claim 1, wherein the cavity is cylindrical.

5. The phantom of claim 1, wherein one or more of the rods extend partially or entirely through a core region of the cavity, the core region extending across the full extent of the cavity in the z-direction of the phantom, but only partway across the cavity in the cross-sectional plane of the phantom.

6. The phantom of claim 4, wherein the core region is cylindrical and concentric with the cavity and extends no more than 50% across the diameter of the cavity.

7. The phantom of claim 1, comprising a fluid in the cavity.

8. The phantom of claim 1, wherein the rod cross-sections are of different sizes or shapes from one another.

9. The phantom of claim 8, wherein one rod cross-section has a size corresponding to the expected spatial resolution limit of an imaging apparatus.

10. The phantom of claim 1, wherein the rods comprise a sealed radioactive material.

11. The phantom of claim 1, wherein one or more of the rods comprises a bore, extending along the elongation direction of the rod, the bore being for holding a fluid therein.

12. The phantom of claim 11, wherein the bore is lined with capillary tubing.

13. The phantom of claim 11, comprising a fluid in the bore of the rod.

14. The phantom of claim 13, wherein the fluid is radioactive.

15. A phantom for determining the alignment of two or more imaging apparatuses, the phantom comprising a housing, and two or more conduits extending through the housing, the conduits being for holding a fluid therein, the conduits being non-parallel with one another.

16. The phantom of claim 15, wherein the housing comprises a solid block of material, and the conduits are provided by bores in the block of material.

17. The phantom of claim 16, wherein the material is polystyrene.

18. The phantom of claim 16, wherein more than two conduits, more than three conduits, more than four conduits or more than five conduits are provided that extend through the housing.

19. The phantom of claim 16, wherein all the conduits are non-parallel with one another.

20. The phantom of claim 16, wherein the housing is cylindrical.

21. The phantom of claim 16, wherein one or more of the conduits extend partially or entirely through a core region of the housing, the core region extending across the full extent of the housing in the z-direction of the phantom, but only partway across the cavity in the cross-sectional plane of the phantom.

22. The phantom of claim 20, wherein the core region is cylindrical and concentric with the housing and extends no more than 50% across the diameter of the housing.

23. The phantom of claim 15, comprising fluid in the conduits.

24. The phantom of claim 15, wherein the conduit cross-sections are of different sizes or shapes from one another.

25. The phantom of claim 24, wherein one conduit cross-section has a size corresponding to the expected spatial resolution limit of an imaging apparatus.

26. The phantom of claim 15, wherein the conduits are straight and cylindrical.

27. (canceled)

28. A method of determining the alignment of two or more imaging apparatuses, the method comprising the steps of:
providing a phantom according to claim 1,
generating respective cross-sectional images of the phantom using each imaging apparatus, the rod cross-sections or conduit cross-sections being shown in the images; and
determining, from the positioning of the rod/conduit cross-sections, the relative alignment of the images in the z-direction and cross-sectional plane of the phantom.

29. The method of claim 28, wherein a range for the spatial resolution of one or both of the imaging apparatuses is calculated by determining which rod/conduit cross-sections can and cannot be seen in the images.
